(19) United States
(12) Patent Application Publication
(10) Pub. No.: US 2009/0149860 A1
(43) Pub. Date: Jun. 11, 2009

(54) DEVICE FOR DELIVERY OF BONE VOID FILLING MATERIALS

(76) Inventors: Robert M. Scribner, Boulder, CO (US); Lawrence R. Jones, Conifer, CO (US)

Correspondence Address: Moore & Hansen, PLLP, 225 South Sixth Street, Suite 4850, Minneapolis, MN, 55402, US

(21) Appl. No.: 11/994,837

(22) PCT Filed: Jul. 7, 2006
(86) PCT No.: PCT/US06/26616

§ 371 Date: Nov. 10, 2008

Related U.S. Application Data
(60) Provisional application No. 60/697,146, filed on Jul. 7, 2005.

(51) Int. Cl. A61B 17/58

(52) U.S. Cl. 606/93; 606/92; 606/94

(57) ABSTRACT

Delivery devices and methods to be used in the delivery of filling/stabilizing and/or therapeutic materials to a bone or other targeted anatomical site. The disclosed devices desirably control the flow of material, measure the volume of material delivered to the site of interest, and prevent the placement of materials in unintended locations. These disclosed devices desirably combine multiple uses of mechanical control of the filler/stabilizing material delivery, rotational advancement of syringe mechanisms in combination with the advantages of manual displacement of filling/stabilizing material from a cannula/stylet pair.

### CROSS-REFERENCE TO RELATED APPLICATION

This application claims the benefit of U.S. Provisional Patent Application Ser. No. 60/697,146, filed 7 Jul. 2005, entitled “Device for Delivery of Bone Void Filling Materials,” the disclosure of which is incorporated herein by reference.

### FIELD OF THE INVENTION

The invention generally relates to the treatment of bones and/or other anatomical regions in humans and other animals.

### BACKGROUND OF THE INVENTION

The creation of voids within bones and/or other body regions, including the deployment of expanding structures (i.e., balloons) or bone cutting instruments (i.e., drills and/or osteotomes) to create such voids, is well known in the art. As part of a fracture fixation procedure or other surgical intervention, a filling and/or stabilizing material, such as bone cement or other therapeutic compound, may desirably be introduced into a targeted void (or other location) within bone or other body regions to stabilize, support, repair and/or augment the targeted anatomical structure. Several companies offer bone cement injection devices. These devices are similar to a household caulking gun. Typically, the injection device has a pistol-shaped body, which supports a cartridge containing bone cement. The cement is typically in two or more parts (i.e., components) and must be mixed in a mixer and transferred into the cartridge for injection (or is mixed in the cartridge itself prior to or during injection). Just after mixing, and prior to curing, the filling/stabilization material is in a flowing, viscous liquid state, similar to a syrup or watery pancake batter in consistency. The injection device generally incorporates a ram or other flow inducing device, which is actuated by a manually movable trigger/screwing mechanism to push the viscous filling/stabilization material out the front of the cartridge through a suitable nozzle and into the interior of a bone targeted for treatment. Once injected into the targeted bone, the filling/stabilization material undergoes a curing cycle of perhaps six to eight minutes. While curing, the filling/stabilization material passes from a viscous liquid to a putty-like consistency and finally to a hard rigid block.

### SUMMARY OF THE INVENTION

Embodiments of the invention may provide greater control over the placement of filling/stabilization material (such as PMMA bone cement) and/or other flowable liquids/materials into bone and/or other body regions, including the ability to introduce filler and/or stabilizing materials in a plurality of controlled manners without requiring removal/replacement of the injection device during the procedure. Embodiments may also facilitate the injection of highly viscous filling/stabilizing material into the bone or other tissues, either into a cavity formed within the bone or other tissues, or directly into the bone or other tissues themselves.

Features and advantages of various embodiments of the invention are set forth in the following Description and Drawings, as well as in the appended Claims.

### BRIEF DESCRIPTION OF THE DRAWINGS

FIG. 1 is a lateral view of a human spinal column;

FIG. 2 is a top plan view, with portions broken away, of a human vertebral body;

FIG. 3 is a lateral view, with portions broken away of a series of vertebral bodies;

FIG. 4 is a perspective view of one embodiment of an introduction device constructed in accordance with various teachings of the invention;

FIG. 5 is another perspective view of the introduction device of FIG. 4;

FIG. 6 is a cut-away side view of the introduction device of FIG. 4;

FIG. 7 is a partial cut-away side view of the introduction device of FIG. 4;

FIG. 8 is another partial cut-away side view of the introduction device of FIG. 4.

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

### DETAILED DESCRIPTION OF THE PREFERRED EMBODIMENTS

This Specification describes new systems and methods to facilitate the introduction of filling, stabilizing and/or therapeutic materials to treat bones and other anatomical structures. It should also be appreciated that the new systems and methods can be utilized to treat bones without use of any cavity-creation and/or fracture reduction devices, if desired. The new systems and methods will be described with regard to the treatment of vertebral bodies. It should be appreciated, however, that the systems and methods so described are not limited in their application to vertebrae. The systems and methods are applicable to the treatment of diverse bone types, including, but not limited to, such bones as the radius, the humerus, the femur, the tibia or the calcaneus, as well as other anatomical regions of human or animal bodies.

### Vertebral Bodies

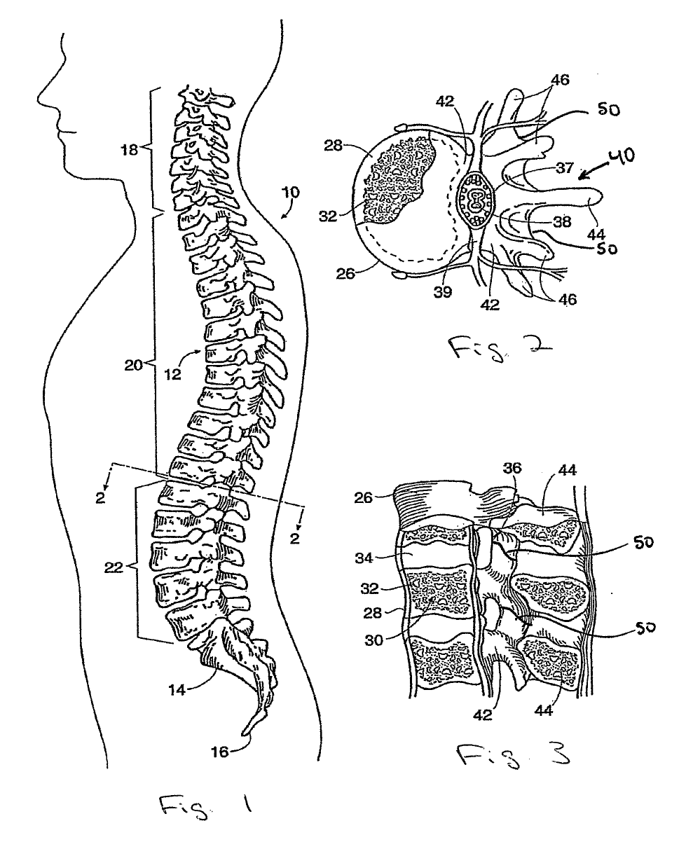

As FIG. 1 shows, the spinal column 10 comprises a number of uniquely shaped bones, called the vertebrae 12, a sacrum 14, and a coccyx 16 (also called the tail bone). The number of vertebrae 12 that make up the spinal column 10 depends upon the species of animal. In a human (which FIG. 1 shows), there are twenty-four vertebrae 12, comprising seven cervical vertebrae 18, twelve thoracic vertebrae 20, and five lumbar vertebrae 22. When viewed from the side, as FIG. 1 shows, the spinal column 10 forms an S-shaped curve. The curve serves to support the head, which is heavy. In four-footed animals, the curve of the spine is simpler.

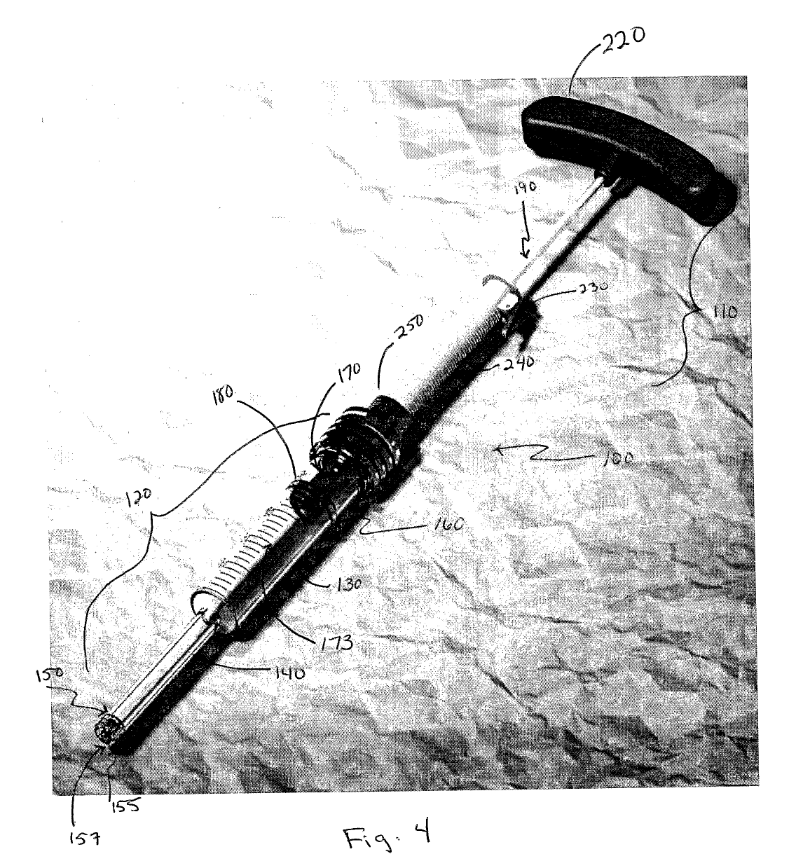

As FIGS. 1 to 3 show, each vertebra 12 includes a vertebral body 26, which extends on the anterior (i.e., front or chest) side of the vertebra 12. The vertebral body 26 is in the shape of an oval disk. The vertebral body 26 includes an exterior formed from compact cortical bone 28. The cortical bone 28 encloses an interior volume 30 of reticulated cancellous, or spongy, bone 32 (also called medullary bone or trabecular bone). A “cushion,” called an intervertebral disk 34, is located between the vertebral bodies 26.

An opening, called the vertebral foramen 36, is located on the posterior—i.e., back) side of each vertebra 12. The spinal ganglion 39 pass through the foramen 36. The spinal cord 38 passes through the spinal canal 37. The vertebral arch 40 surrounds the spinal canal 37. The pedicle 42 of the vertebral arch 40 adjoins the vertebral body 26. The spinous process 44 extends from the posterior of the vertebral arch 40, as do the left and right transverse processes 46.

### Treatment of Vertebral Bodies

### Access to Targeted Anatomical Regions

Access to a vertebral body (or any other targeted anatomical regions) can be accomplished from many different directions, depending upon the targeted location within the vertebral body, the intervening anatomy, and the desired complexity of the procedure. For example, access can also be obtained through one or more pedicles (transpedicular), outside of a pedicle (extrapedicular), along either side of the vertebral body (posterolateral), superiorly or inferiorly (such as through an upper or lower intervertebral disc and/or adjacent vertebral bodies), laterally and/or anteriorly. In addition, such approaches can be used with a closed, minimally invasive procedure or with an open procedure (or some combination thereof).

### Instruments for Establishing Bone Access and Passage Creation

During a typical bilateral procedure to access a targeted vertebral body, a patient lies on an operating table. The patient can lie face down on the table, or on either side, or at an oblique angle, depending upon the physician's preference. The physician initially introduces a spinal needle assembly into soft tissue in the patient's back. Under radiologic or CT monitoring, the physician advances the spinal needle assembly through soft tissue down to and into the targeted vertebral body. The physician can also employ stereotactic instrumentation to guide advancement of the spinal needle assembly and subsequent tools during the procedure. In this arrangement, the reference probe for stereotactic guidance can be inserted through soft tissue and implanted on the surface of the targeted vertebral body. The entire procedure can also be monitored using tools and tags made of non-ferrous materials, e.g., plastic or fiber composites, such as those disclosed in U.S. Pat. Nos. 5,782,764 and 5,744,958, which are each incorporated herein by reference, which would be suitable for use in a computer enhanced, whole-room MRI environment.

The physician will typically administer a local anesthetic, for example, lidocaine, through the spinal needle assembly. In some cases, the physician may prefer other forms of anesthesia. The physician directs the spinal needle assembly to penetrate the cortical bone and/or cancellous bone through the side of the vertebral body. Preferably the depth of penetration is about 60% to 95% of the vertebral body. The physician holds the stylus and withdraws the stylet of the spinal needle assembly. The physician then slides a guide pin instrument through the stylus and into the cancellous bone. The physician now removes the stylus, leaving the guide pin instrument deployed within the cancellous bone.

The physician next slides an obturator instrument over the guide pin instrument, distal end first. The physician can couple the obturator instrument to a handle, which facilitates manipulation of the instrument. The physician makes a small incision in the patient's back. The physician twists the handle while applying longitudinal force to the handle. In response, the obturator instrument rotates and/or penetrates soft tissue through the incision. The physician may also gently tap the handle, or otherwise apply appropriate additional longitudinal force to the handle, to advance the obturator instrument through the soft tissue along the guide pin instrument down to the cortical bone entry site. The physician can also tap the handle with an appropriate striking tool to advance the obturator instrument into a side of the vertebral body to secure its position.

The obturator instrument has an outside diameter that is generally well suited for establishing a lateral access. However, if access is desired through a narrower region of the vertebral body, e.g., a pedicle (generally called a transpedicular access), the outside diameter of the obturator instrument can be reduced to well below the diameter of the pedicle. The reduced diameter of the obturator instrument mediates against damage or breakage of the pedicle. It should be understood that the disclosed methods and devices are well suited for use in conjunction with other approach paths, such as pedicular, extra-pedicular, posterolateral and anterior approaches, with varying results. The physician then proceeds to slide the handle off the obturator instrument and to slide a cannula instrument over the guide pin instrument and, further, over the obturator instrument. If desired, the physician can also couple a handle to the cannula instrument, to apply appropriate twisting and longitudinal forces to rotate and advance the cannula instrument through soft tissue over the obturator instrument. When the cannula instrument contacts cortical bone, the physician can appropriately tap the handle with a striking tool to advance the end surface of the cannula into the vertebral body to secure its position.

The physician now withdraws the obturator instrument, leaving the cannula instrument in place. When a reduced or tapered diameter obturator instrument is used, the physician can remove an inner centering sleeve. The physician slides a drill bit instrument through the cannula instrument, until contact between the machined or cutting edge of the drill bit instrument and cortical bone occurs. The physician then couples the drill bit instrument to the handle. Guided by X-ray (or another external/non-invasive and/or partially invasive visualizing system), the physician applies appropriate twisting and longitudinal forces to the handle, to rotate and advance the machined edge of the drill bit instrument to open a lateral passage through the cortical bone and into the cancellous bone. The drilled passage preferably extends no more than 95% across the vertebral body.

### Additional Passage/Osteotomy Plane Creation

If desired, once access has been established, the physician may desire to create additional passages and/or one or more osteotomy planes within the targeted anatomical structure. Such additional passage/osteotomy plane(s) may be created within the bone and/or targeted anatomical structure(s) utilizing expandable structures (i.e., balloons) and/or bone cutting or manipulating instruments that can desirably be passed through one or more cannula instrument(s). Various devices and methods of using such devices is described in copending application entitled “Devices and Methods for the Treatment of Bone Fracture,” filed concurrently, the disclosure of which is incorporated herein by reference.

### Introduction of Filling/Stabilizing/Therapeutic Materials

Once the passage, any additional passage(s) and/or one or more osteotomy planes have been created in the vertebral body (or if no such passage is formed or deemed necessary, as desired by the physician), the physician can introduce a filling/stabilizing and/or therapeutic material into the targeted vertebral body (or other anatomical location). The material can desirably comprise a material that resists torsional, tensile, shear and/or compressive forces within the passage, thereby providing renewed interior structural support for the surrounding cancellous and/or cortical bone, stabilize, secure and/or encapsulate cancellous and/or cortical bone fragments and/or serve as a source of therapeutic materials to treat medical conditions within and/or outside of the treated anatomical location. For example, the material can comprise a flowable material, such as bone cement, allograft tissue, autograft tissue, hydroxyapatite or other natural or synthetic bone substitute, which can be introduced into the passage and which, in time, sets to a more-hardened (desirably load-bearing and/or more stabilized) condition. The material can also comprise a compression-resistant material, such as rubber, polyurethane, cyanoacrylate, or silicone rubber, which is inserted into the passage. The material can also comprise a semi-solid slurry material (e.g., a bone slurry in a saline base), which is either contained within a porous fabric structure located in the passage or injected directly into the passage, to resist compressive forces within the passage. Alternatively, the material could comprise stents, reinforcing bar (“Re-Bar”) or other types of internal support structures, which desirably resist compressive, tensile, torsional and/or shear forces acting on the bone and/or filler/stabilizing/therapeutic material. The filling/stabilizing material may also comprise medication, or a combination of medication and compression-resistant material, as described above.

Alternatively, the filling/stabilizing material can comprise a bone filling/stabilizing material which does not withstand compressive, tensile, torsional and/or shear forces within the cavity, or which does not immediately stabilize one or more of the surrounding bone fragments. For example, where the patient is not expected to experience significant forces within the spine immediately after surgery (i.e., the patient is confined to bed rest or wears a brace), the filling/stabilizing material need not be able to immediately bear loads. Rather, the filling/stabilizing material could provide a scaffold for bone growth, or could comprise a material which facilitates or accelerates bone growth, allowing the bone to heal over a period of time. As another alternative, the filling/stabilizing material could comprise a resorbable or partially-resorbable source of organic or inorganic material for treatment of various bone or non-bone-related disorders including, but not limited to, osteoporosis, cancer, degenerative disk disease, heart disease, acquired immune deficiency syndrome (AIDS) or diabetes. In this way, the cavity and/or filler/stabilizing material could comprise a source of material for treatment of disorders located outside the treated bone.

### Material Containment Vessel

In one alternative embodiment, the physician may choose to utilize the invention in combination with a material containment vessel, such as the vessel disclosed in the copending application entitled “Devices and Methods for the Treatment of Bone Fracture,” filed concurrently, the disclosure of which is incorporated herein by reference. Following injection of the filling/stabilizing material, the vessel may be removed from the targeted bone, or can be left in the cavity, as desired by the physician. In such an arrangement, the filling/stabilizing material would be dispensed from the introduction device into the vessel, which desirably serves to contain the filling/stabilizing material and/or isolate the filling/stabilizing material from surrounding tissues for a period of time. Alternatively, the vessel, filled with the filling/stabilizing material, could serve to provide interior structural support function for the surrounding cancellous and/or cortical bone (if removal of the vessel is not desired by the physician). In various embodiments, the vessel could comprise an inert, durable, non-degradable plastic material, e.g., polyethylene and/or other polymers. Alternatively, the vessel could comprise an inert, bio-absorbable material, which degrades over time for absorption or removal by the body.

Desirably, the filling/stabilizing material or one of its components can additionally serve as an expansion medium for the vessel, allowing for the compaction of cancellous bone and/or formation of a passage, cavity and/or one or more osteotomy planes, and eventually perform compaction, interior support and/or stabilization functions. In an alternative embodiment, the vessel can be first expanded/enlarged with an expansion medium (i.e., to create a passage, cavity and/or osteotomy plane within the targeted anatomical region), and the filling/stabilization material can be subsequently introduced (either after the expansion material is removed or, if the expansion material forms a component of the filling/stabilizing material, concurrent with and/or after the expansion medium is introduced). In one alternative embodiment, the filling/stabilizing material could comprise a two-part material including, but not limited to, settable polymers or calcium alginate.

The vessel can comprise a permeable, semi-permeable, or porous material, which allows the transfer of medication or other materials through the wall of the vessel. If desired, the vessel can comprise a membrane that allows osmotic and/or particulate transfer through the vessel, or the vessel can comprise a material that allows the medication to absorb into and/or diffuse through the vessel. Alternatively, medication can be transported through a porous wall material by creating a pressure differential across the wall of the vessel, or fluids, cells and/or other materials from the patient's body can pass and/or be drawn through the vessel wall and into the vessel for various purposes including, but not limited to, fluid/cellular analysis, bony ingrowth, bone marrow harvesting, and/or gene therapy (including gene replacement therapy).

### Injection and/or Introduction of Materials

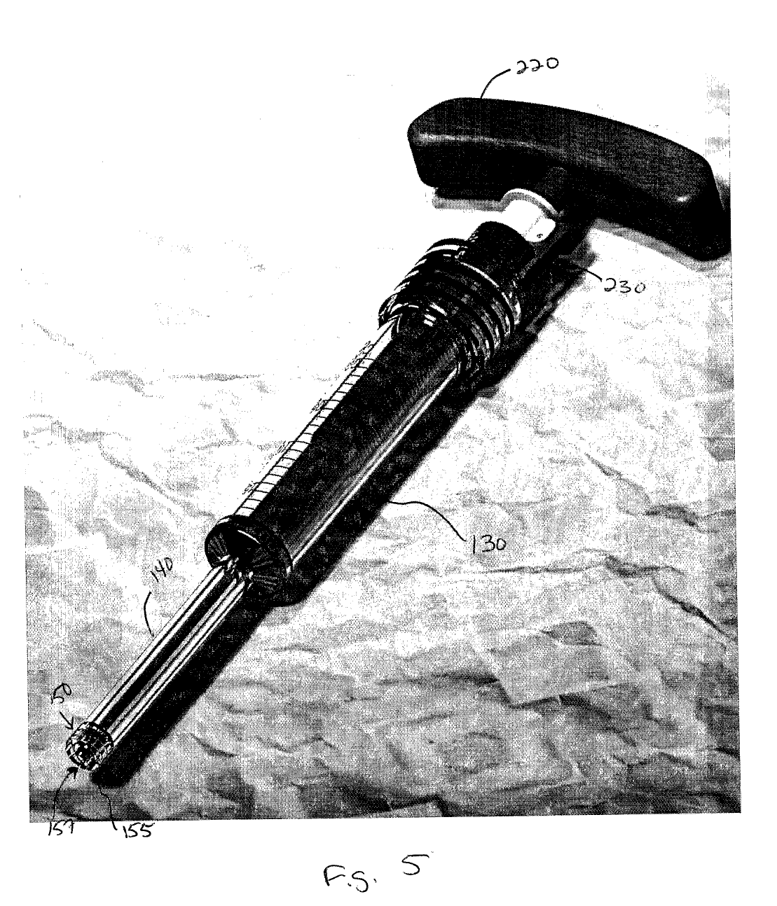

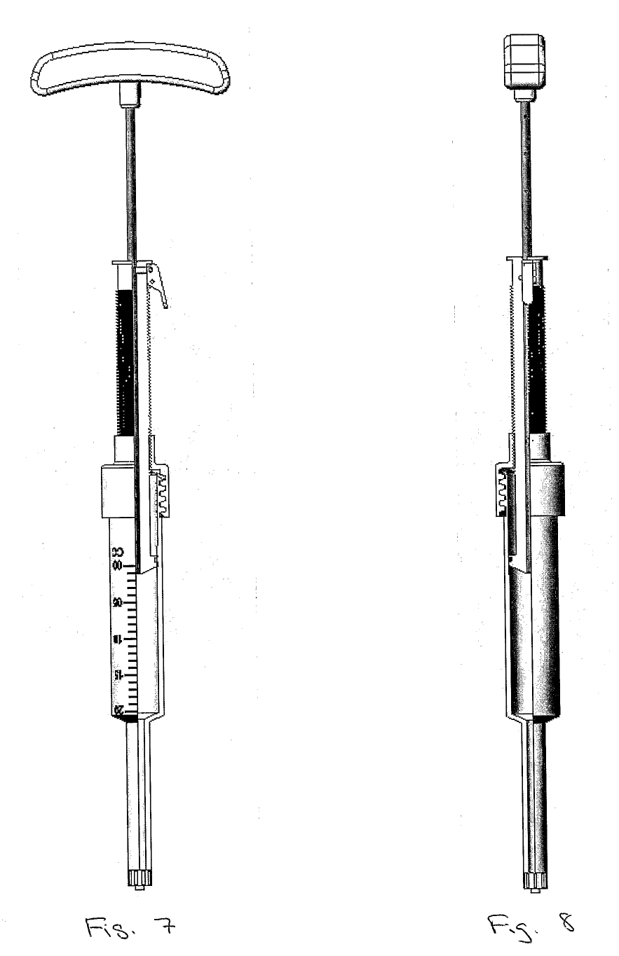

FIGS. 4-8 depict one embodiment of a material introduction device constructed in accordance with various teachings of the invention. Desirably, this device facilitates the controlled injection of desired amounts of filling/stabilizing material into a bone or other vertebral body (including into a vessel or other device contained therein). The introduction device 100 comprises a two-stage plunger 110 coupled to a two-stage material reservoir 120. The two-stage reservoir 120 comprises a first section 130 and a second section 140. The first and second sections 130 and 140 are desirably hollow tubular bodies connected and/or secured in a sealing relationship, with the interior of the first section 130 being in fluid communication with the interior of the second section 140. The first section 130 has a first interior cross-sectional area, and the second section 140 has a second interior cross-sectional area. Desirably, the first interior cross-sectional area will be greater than the second interior cross-sectional area. If also desired, the transition from the first section 130 to the second section 140 can neck down or taper.

In the disclosed embodiment, the first section 130 comprises a cylindrical, hollow, tubular member having an interior diameter and a length as determined by the application, and the second section 140 comprises a cylindrical, hollow, tubular member having an interior diameter and a length as determined by the application. A luer fitting 150 (or other suitable connection device) is positioned around a dispensing opening 157 at the distal tip 155 of the second section 140. In one embodiment, the luer fitting 150 is desirably sized and configured to dock or mate with a corresponding fitting (not shown) on a corresponding catheter attached to a material containment vessel (not shown). Of course, the distal tip 155 could alternatively incorporate a dispensing opening 157 without a luer fitting 150, or possibly an atraumatic dispensing tip or similar docking mechanism, if desired. Alternatively, the distal tip can be designed to have a conical or tapered end for easy advancement and immediate use for injecting directly into bone or other targeted anatomical structure(s). The distal tip may also be designed to have radiopaque markers for easy positioning and placement during use of indirect/non-invasive and/or minimally invasive visualization techniques known in the art.

The two-stage plunger 110 comprises a first ram or plunger 170, positioned within the first section 130, which is sized and configured to pass through the interior of the first section 130. A seal 160, such as an O-ring, is secured to the distal end 180 of the first plunger 170 in a manner well known in the art. Desirably, the seal 160 will slidingly engage with the inner walls of the first section 130 to seal the proximal end of the first section 130 as the first plunger 170 advances therethrough. The seal 160 can comprise polytetrafluoroethylene (PTFE, also known under the tradename of “Teflon”), natural rubber, or other type of known sealant material. The seal can also be coated with a hydrophilic or hydrophobic material that facilitates easy translation through the first section 130. It should be noted that, while the cross-section of the disclosed plunger appears circular in FIG. 4, plungers having other cross-sectional shapes, such as triangular or rectangular shapes, could similarly be utilized. An opening 173 is formed in the central area of the seal 160.

If desired, the plunger and cylinder wall materials can be designed to withstand the linear forces and/or temperatures of the exothermal or chemical reagents of other biochemical reaction(s) of the mixed filler/stabilizing material without bending, breaking or otherwise significantly degrading, such as hard plastics or metals. The inner diameter of the cylinder(s) can also be coated with the same hydrophilic or hydrophobic material to allow easy translation of the seal. The external body of the first plunger can include or have printed graduations for measurements or amount of material injected as commonly used in the art.

The two-stage plunger 110 further comprises a second ram or plunger 190, at least a portion of which is sized and configured to extend through a lumen 200 formed within the first plunger 170 (as well as in the opening 173 formed in the seal 160). A T-handle 220 or other suitable manipulating device is positioned at a proximal end of the second plunger 190. In another embodiment, the T-handle 220 and remainder of the device can be designed to have a lumen accommodating a guidewire for easy positioning and/or placement and/or removal of the guidewire through the device. The second plunger 190 desirably extends through the lumen 200 such that the distal end of the second plunger sits approximately flush with the opening 173 in the seal 160. One or more securement or latching mechanisms 230 are positioned on the first plunger 170, desirably engaging a corresponding detent or notch 210 or other engagement mechanism on the second plunger 190. When engaged, the latching mechanism 230 secures the first and second plungers 170 and 190 together, but when disengaged, the second plunger 190 may be advanced longitudinally through the lumen 200 of the first plunger 170. If desired, the shaft of the second plunger 190 and the corresponding lumen 200 need not be circular or cylindrical in shape, but rather may be a range of shapes that prevent and/or inhibit relative rotation between the lumen 200 and the second plunger 190, yet desirably allow longitudinal movement of the second plunger 190 when the latching mechanism 230 is released.

Desirably, the shape and size of the cross-sectional area of the second plunger 190 will approximate the shape and size of the cross-sectional area of the second section 140, such that the second plunger is capable of displacing substantially all of the filler/stabilizing material in the second section as the second plunger advances. Similarly, the shape and size of the cross-sectional area of the first plunger 170 (or that of the seal 160) will desirably approximate the shape and size of the cross-sectional area of the first section 130, such that the first plunger is capable of displacing substantially all of the filler/stabilizing material in the first section as the first plunger advances. In an alternative embodiment, the second plunger can be designed to accommodate a guidewire for easy positioning and/or placement of the device. For material strength, the various components of the introduction device 100 can comprise a substantially rigid metal, plastic or ceramic material (i.e., stainless steel or a high strength plastic) or some combination thereof.

The first plunger 170 further incorporates a series of external screw threads 240, which engage with corresponding internal screw threads 250 within the first section 130. Desirably, the corresponding sets of screw threads 240 and 250 are sized and configured such that, in the disclosed embodiment, one complete rotation of the first plunger would result in the expellation of approximately 1 cc of material contained within the first section 130. Of course, other thread designs and arrangements could be utilized to allow other defined controlled injection amounts. The threads desirably provide a mechanical “force multiplication” advantage, converting the rotation of the T-handle and plungers to a longitudinal advancement of the first plunger within the first section and a commensurate dispensing of filler/stabilizing material. The first cylinder surrounding the first plunger can also be designed to be interchangeable or dispensable in case filler/stabilizing material hardens and/or cures—allowing a new cylinder to be introduced to replace one containing hardened filler/stabilizing material and used accordingly. Alternatively, the body of the first plunger may also incorporate a one-way valve or seal that prevents the retrograde flow of blood and/or other body fluids, yet still allows translation of the second plunger 190 through the extended nose or body of the second section 140. If desired, a side port or relief valve can be integrated into the device to release any trapped air that may be contained within the filler/stabilization material or materials that may embolize and enter the device during injection of the filler/stabilizing material. The air port could accommodate a seal, one-way valve or cap to cover and/or eliminate expellation and/or introduction of air or other gases and/or fluids.

When injection of filler/stabilizing material is desired, the first and/or second sections 130 and/or 140 of the introduction instrument 100 can be filled with filler/stabilizing material (not shown) such as bone cement or PMMA. As the T-handle is rotated in a clockwise direction, the first and second plungers 170 and 190 rotate together, advancing the first plunger 170 through the first section 130 and expelling filler/stabilizing material in the first section 170 through the second section 140 and out the dispensing opening 157. Passage of a significant amount of filler/stabilizing material back through the opening 173 in the central area of the seal 160 is desirably prevented by the presence of the second plunger 190 in the opening 173.

When a desired amount of filling/stabilizing material has been expelled, or thickening/polymerization of the filler/stabilizing material has rendered the material within the device so resistant to flow that rotation of the T-handle is no longer safe or feasible, it may still be desirous to expel an additional metered amount of filler/stabilizing material to ensure the clinical objectives. In such a case, the illustrated embodiment allows the physician to disengage the latching mechanism 230, and the second plunger 190 may then be pushed into and through the first section 130, desirably expelling an additional amount of filler/stabilizing material equal to the volume of the second plunger entering the first section 130. Further advancement of the second plunger 190 in this manner will allow the second plunger to eventually enter and displace filler/stabilizing material within the second section 140, which in the disclosed embodiment would desirably amount to an additional 1-1.5 cc of filler/stabilizing material.

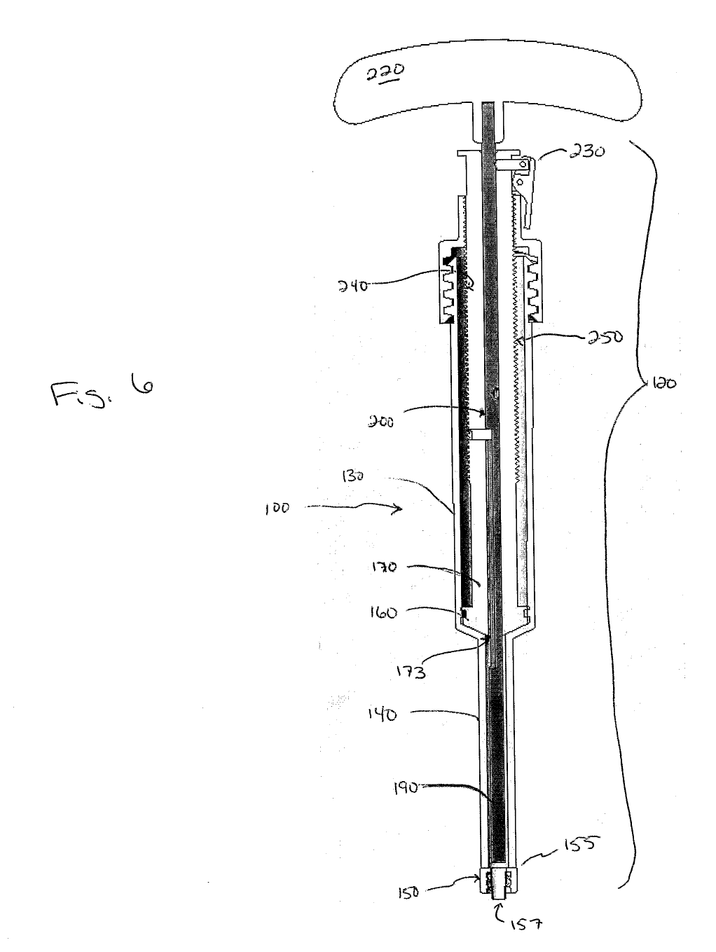

If desired, the second plunger 190 may be of a length sufficient to travel to the end of the dispensing opening 157 (see FIG. 5). Alternatively, the second plunger 190 could be of a significantly longer length, potentially allowing the distal tip of the second plunger 190 to travel through and past the dispensing opening 157, ultimately into the surgical instruments used to access the targeted anatomical structures (i.e., catheter tubing and/or minimally-invasive cannulae) and/or into the anatomical structures themselves. In such an arrangement, the second plunger could potentially be utilized to tamp and evacuate any residual filler/stabilizing material still within the cannula, and/or manipulate filling/stabilizing material within the targeted anatomical region.

If desired, the second plunger may incorporate additional detents along its length such that, during advancement of the second plunger, the latching mechanism causes an audible or tactile “click” to indicate the approximate position of the advancing second plunger relative to the latching mechanism, or that can temporarily “lock” the latching mechanism to prevent accidental withdrawal of the second plunger.

By utilizing first and second sections of different cross-sectional areas, and first and second plungers to displace the filler/stabilizing material, the illustrated embodiment facilitates dispensing of a substantial amount of filler/stabilizing material from a single introduction device. Because the viscosity of PMMA and various other types of filler/stabilizing materials typically increases with time during the dispensing process, it becomes progressively harder to dispense filler/stabilizing material over time. By utilizing a plunger of larger cross-sectional area to initiate the filling operation, when the filler/stabilizing material is less viscous, the illustrated embodiment allows dispensing of a significant amount of filler/stabilizing material through rotation of the T-handle. However, as the filler/stabilizing material cures, and becomes more viscous, the reduced cross-sectional area of the second plunger allows continued dispensing of the more viscous filler/stabilizing material, even when it is in a highly viscous state. Moreover, because the second section is of reduced cross-sectional area, its reduced profile will desirably allow the distal tip of the filler instrument to be introduced through a cannula and/or soft tissues and directly into a targeted vertebral body, while still providing a sufficient reservoir of filler/stabilizing material to accomplish the goals of augmenting/stabilizing and/or repairing the targeted bone.

Moreover, because the introduction device need not be refilled and/or “switched out” during the dispensing operation, and can remain in place and dispense the entire required amount of bone filler/stabilizing material for the procedure, the potential for trapping air within the vertebral body and/or bolus of filler/stabilizing material is significantly reduced. The illustrated embodiment also greatly facilitates the ability of the physician to immediately shift from a higher volume, lower pressure filler/stabilizing material flow to a lower volume, higher pressure filler/stabilizing material flow. As the first plunger is being advanced, and filler/stabilizing material is being injected into the vertebral body, the physician may determine that a more controlled, higher pressure and/or lower volume flow of filler/stabilizing material is needed. Alternatively, the filler/stabilizing material may cure or harden to a point where further movement of the first plunger is extremely difficult and/or impossible to effect. One embodiment of the invention permits the physician to advance the second plunger into the second section, even when the distal end of the first plunger is not adjacent, near and/or abutting the distal end of the first section. As the second plunger passes through the filler/stabilizing material in the first section, and enters the second section, filler/stabilizing material will desirably continue to be displaced from the dispensing opening. Due to the decreased cross-sectional area of the second plunger and second section (as compared to the first plunger and first section), the second plunger may more easily be pushed through the filling/stabilizing material in the first section and filling/stabilizing material can more easily be dispensed from the second section at higher pressures and/or lower volumes.

The disclosed introduction device may be used to introduce filler/stabilizing material through a cannula into a cavity created within a bone, or may be used with vertebroplasty-type techniques to introduce filler/stabilizing material directly into the vertebral body without prior formation of a cavity. Where prior cavity-formation is not required and/or desired, and vertebroplasty-like techniques will be used, the filler instrument can incorporate a needle-point at the distal end of the instrument, or the diameter of the second section can be significantly reduced to allow passage of the instrument (or the second plunger, if desired) through the lumen of a spinal needle assembly.

Alternatively, one or more of the sections of the introduction device could comprise a commercially available spinal needle assembly (such as a Bone Marrow Biopsy Needle, available from Becton Dickinson & Co., Franklin Lakes, N.J., 07417 USA). If desired, one or more plunger assemblies of varying sizes and lengths could be provided to accommodate differing spinal needle assemblies.

If desired, the introduction device can be pre-loaded with filler/stabilizing material, introduced through soft tissues and into the targeted vertebral body, used to inject filler/stabilizing material, and removed, quickly and easily without need for tool exchanges during the operation. For example, where the end plates of the vertebral body have depressed to a point where a cavity cannot be safely created within the vertebral body (i.e., through the use of bone manipulating instruments and/or expandable structures), filling/stabilizing material can be introduced under pressure through a needle directly into the cancellous bone of the vertebral body. The filler/stabilizing material desirably penetrates cancellous bone.

The delivery of bone void filling/stabilizing materials to anatomic locations can be accomplished by direct placement to the void site in an open surgical setting, or by percutaneous delivery to the void site by means of delivery devices. The size and shape of any devices disclosed herein are desirably selected by the physician, taking into account the morphology and/or geometry of the site to be treated. The shape of the joint, the bones and soft tissues involved, and the local structures that could be harmed if moved inappropriately, are generally understood by medical professionals using textbooks of human anatomy along with their knowledge of the site and its disease and/or injury. The physician is also desirably able to select the desired shape and/or size of instrument(s) and its/their placement in and/or around the joint (or other anatomical structure) based upon prior analysis of the morphology of the targeted anatomical region using, for example, plain film x-ray, fluoroscopic x-ray, MRI or CT scanning. The shape, size and placement are desirably selected to optimize the strength and ultimate bonding of the filling/stabilizing material to the surrounding bone and/or tissues of the targeted anatomical region.

The systems and methods embodying the invention can be adapted for use virtually in any interior body region, include body regions where the formation of a cavity or void within tissue is or is not required and/or desired for a therapeutic or diagnostic purpose. The preferred embodiments show the invention in association with systems and methods used to treat bones. This is because the systems and methods which embody the invention are well suited for use in this environment.

It should be appreciated that the systems and methods which embody features of the invention can be used in other interior body regions, including other non-bone body regions, which are also contemplated. It should also be understood that the principles of the various embodiments of the invention could be applied by those skilled in the art to a wide variety of mammals and/or other animals, with varying results.

Although the disclosure hereof is detailed and exact to enable those skilled in the art to practice the invention, the physical embodiments herein disclosed merely exemplify the invention that may be embodied in other specific structures. Other embodiments and uses of the invention will be apparent to those skilled in the art from consideration of the specification and practice of the invention disclosed herein. All documents referenced herein are specifically and entirely incorporated by reference. The specification and examples should be considered exemplary only with the true scope and spirit of the invention indicated by the following claims. As will be easily understood by those of ordinary skill in the art, variations and modifications of each of the disclosed embodiments, including combinations or the various embodiments disclosed herein, can be easily made within the scope of this invention as defined by the following claims.

We claim:

1. An introduction device for controlled injection of desired amounts of filling/stabilizing material into an anatomic structure, comprising:
a two-stage plunger coupled to a two-stage material reservoir;
in which the two-stage material reservoir comprises a first section and a second section, each comprising a hollow tubular body sealingly connected to the other such that an interior of the first section is in fluid communication with an interior of the second section;
the two-stage plunger comprising a first plunger, positioned within the first section, sized and configured to pass through an interior of the first section; and a second plunger, positioned within the second section, sized and configured to pass through an interior of the second section;
a seal secured to a distal end of the first plunger;
the first and second plungers being movable together to advance the first plunger through the first section and expel the filler/stabilizing material in the first section through the second section and out a dispensing opening.

2. The device of claim 1, in which the first section has a first interior cross-sectional area, and the second section has a second interior cross-sectional area, and the first interior cross-sectional area is greater than the second interior cross-sectional area.

3. The device of claim 1, further comprising a connection fitting positioned around a dispensing opening at a distal tip of the second section.

4. The device of claim 1, further comprising a dispensing opening at a distal tip of the second section.

5. The device of claim 1, further comprising an atraumatic dispensing tip at a distal end of the second section.

6. The device of claim 1, further comprising a conical distal tip of the second section that is configured for direct injection into the anatomic structure.

7. The device of claim 1, in which the seal slidingly engages with the interior of the first section to seal the proximal end of the first section as the first plunger advances therethrough.

8. The device of claim 1, in which the cross-section of the plunger is one of appears circular, triangular, and rectangular.

9. The device of claim 1, in which the device further comprises a lumen adapted for at least one of positioning, placement, and removal of a guidewire the guidewire through the device.

10. The device of claim 9, in which the second plunger extends through the lumen such that the distal end of the second plunger is approximately flush with the opening in the seal

11. The device of claim 1, further comprising at least one securement mechanism, positioned on the first plunger, engaging a corresponding engagement mechanism on the second plunger to secure the first and second plungers together, but permit the second plunger to be advanced longitudinally through the lumen of the first plunger when disengaged.

12. The device of claim 11, in which the second plunger and the corresponding lumen are shaped in cross-section to inhibit relative rotation between the lumen and the second plunger, yet allow longitudinal movement of the second plunger when the engagement mechanism is released.

13. The device of claim 1, in which the shape and size of the cross-sectional area of the second plunger approximate the shape and size of the cross-sectional area of the second section, such that the second plunger is capable of displacing substantially all of the filler/stabilizing material in the second section as the second plunger advances.

13. The device of claim 1, in which the shape and size of the cross-sectional area of at least one of the first plunger and the seal approximate the shape and size of the cross-sectional area of the first section, such that the first plunger is capable of displacing substantially all of the filler/stabilizing material in the first section as the first plunger advances.

13. The device of claim 1, in which the second plunger accommodates a guidewire for at least one of positioning and placement of the device.

13. The device of claim 1, in which the first plunger further comprises a series of external screw threads and the first section further comprises a series of corresponding internal screw threads.

14. The device of claim 13, in which the corresponding sets of screw threads are sized and configured such that one complete rotation of the first plunger expels approximately 1 cc of material contained within the first section.

15. The device of claim 14, in which the threads convert rotation of a T-handle and the plungers to longitudinal advancement of the first plunger within the first section and a commensurate dispensing of filler/stabilizing material.

16. The device of claim 1, in which the body of the first plunger further comprises a one-way valve to prevent retrograde flow of body fluids without preventing translation of the second plunger through the body of the second section.

17. The device of claim 1, further comprising a relief valve to release at least one of air contained within the filler/stabilization material and other material that may enter the device during injection of the filler/stabilizing material.

18. The device of claim 1, in which passage of the filler/stabilizing material back through an opening in the central area of the seal is substantially prevented by presence of the second plunger in the opening.

19. The device, of claim 1, in which the second plunger may be pushed into and through the first section to expel an additional amount of filler/stabilizing material equal to the volume of the second plunger entering the first section.

20 The device of claim 1, in which the second plunger has a length sufficient to travel to the end of the dispensing opening

21. The device of claim 1, in which the second plunger has a length sufficient to allow the distal tip of the second plunger to travel through and past the dispensing opening

22. The device of claim 1, in which the second plunger further comprises at least one additional detent along its length such that, during advancement of the second plunger, the latching mechanism indicates approximate position of the second plunger relative to the latching mechanism

23. The device of claim 1, in which the second plunger further comprises at least one additional detent along its length such that, during advancement of the second plunger, the latching mechanism may be locked to prevent accidental withdrawal of the second plunger.
